(19) United States
(12) Patent Application Publication
(10) Pub. No.: US 2025/0118106 A1
(43) Pub. Date: Apr. 10, 2025

(54) STATE ESTIMATION METHOD, STATE ESTIMATION DEVICE, AND RECORDING MEDIUM

(71) Applicant: Panasonic Intellectual Property Management Co., Ltd., Osaka (JP)

(72) Inventors: Shinichi SHIKII, Nara (JP); Aki YONEDA, Hyogo (JP)

(73) Assignee: Panasonic Intellectual Property Management Co., Ltd., Osaka (JP)

(21) Appl. No.: 18/836,200

(22) PCT Filed: Dec. 19, 2022
(86) PCT No.: PCT/JP2022/046648

§ 371 Date: Aug. 6, 2024

(30) Foreign Application Priority Data
Feb. 15, 2022 (JP) 2022-021205

(51) Int. Cl. G06V 40/20; G06V 10/143; G06V 20/52

(52) U.S. Cl. CPC G06V 40/20; G06V 10/143; G06V 20/52

(57) ABSTRACT

A state estimation method includes: capturing a thermal image of at least one imaging target, each of which is a person or an animal, using a thermal imaging camera; obtaining identification information for identifying the at least one imaging target in the thermal image captured in the capturing; and estimating a state of each of at least one estimation target included among the at least one imaging target, based on the thermal image captured in the capturing and the identification information obtained in the obtaining.

### TECHNICAL FIELD

The present invention relates to a state estimation method, a state estimation device, and a program.

### BACKGROUND ART

State estimation methods for estimating at least one of a state of a person or a state of an animal are known. As an example of such a state estimation method, Patent Literature (PTL) 1 discloses a method for identifying a person and an animal based on a signal obtained from a person detecting sensor and then estimating a state of the person.

### CITATION LIST

### Patent Literature

[PTL 1] Japanese Unexamined Patent Application Publication No. 2008-242687

### SUMMARY OF INVENTION

### Technical Problem

Here, a thermal image of at least one imaging target can be captured using a thermal imaging camera to estimate, based on the captured thermal image, a state of at least one estimation target included among the at least one imaging target. In this case, it is difficult to identify the at least one imaging target only from a temperature indicated by the thermal image. Thus, it is also difficult to estimate the state of the at least one estimation target.

In response to the above issue, it is an object of the present disclosure to provide a state estimation method and so forth for easily estimating a state of at least one estimation target.

### Solution to Problem

In accordance with an aspect of the present disclosure, a state estimation method includes: capturing a thermal image of at least one imaging target, each of which is a person or an animal, using a thermal imaging camera; obtaining identification information for identifying the at least one imaging target in the thermal image captured in the capturing; and estimating a state of each of at least one estimation target included among the at least one imaging target, based on the thermal image captured in the capturing and the identification information obtained in the obtaining.

In accordance with another aspect of the present disclosure, a state estimation device includes: an imager that captures a thermal image of at least one imaging target, each of which is a person or an animal, using a thermal imaging camera; an obtainer that obtains identification information for identifying the at least one imaging target in the thermal image captured by the imager; and an estimator that estimates a state of each of at least one estimation target included among the at least one imaging target, based on the thermal image captured by the imager and the identification information obtained by the obtainer.

In accordance with still another aspect of the present disclosure, a program for causing a computer to execute the above-described state estimation method.

### Advantageous Effects of Invention

The present disclosure provides a state estimation method and so forth for easily estimating a state of at least one estimation target.

### BRIEF DESCRIPTION OF DRAWINGS

FIG. 1 is a diagram illustrating a state estimation device and so forth according to Embodiment 1.

FIG. 2 is a block diagram illustrating a functional configuration of the state estimation device illustrated in FIG. 1.

FIG. 3 is a flowchart illustrating an example of an operation performed by the state estimation device illustrated in FIG. 1.

FIG. 4 is a table illustrating a first example of identification information.

FIG. 5 is a diagram illustrating a first example of a thermal image.

FIG. 6 is a table illustrating a second example of the identification information.

FIG. 7 is a diagram illustrating a second example of the thermal image.

FIG. 8 is a table illustrating a third example of the identification information.

FIG. 9 is a diagram illustrating a third example of the thermal image.

FIG. 10 is a table illustrating a fourth example of the identification information.

FIG. 11 is a graph illustrating an example of another operation performed by the state estimation device illustrated in FIG. 1.

FIG. 12 is a diagram illustrating a state estimation device and so forth according to Embodiment 2.

FIG. 13 is a diagram illustrating a state estimation device and so forth according to Embodiment 3.

FIG. 14 is a diagram illustrating a state estimation device and so forth according to Embodiment 4.

FIG. 15 is a diagram illustrating a state management system and so forth according to Embodiment 5.

### DESCRIPTION OF EMBODIMENTS

Hereinafter, certain exemplary embodiments will be described in detail with reference to the accompanying Drawings. The following embodiments are specific examples of the present disclosure. The numerical values, shapes, materials, elements, arrangement and connection configuration of the elements, steps, the order of the steps, etc., described in the following embodiments are merely examples, and are not intended to limit the present disclosure. Among elements in the following embodiments, those not described in any one of the independent claims indicating the broadest concept of the present disclosure are described as optional elements.

Note that the respective figures are schematic diagrams and are not necessarily precise illustrations. Additionally, components that are essentially the same share like reference signs in the figures. Accordingly, overlapping explanations thereof are omitted or simplified.

### Embodiment 1

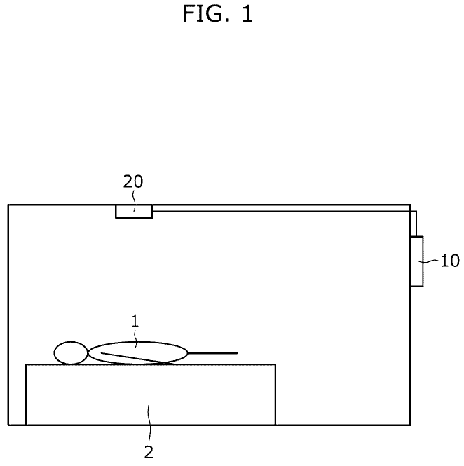

FIG. 1 is a diagram illustrating state estimation device 10 and so forth according to Embodiment 1. State estimation device 10 and so forth according to Embodiment 1 are described with reference to FIG. 1.

As illustrated in FIG. 1, state estimation device 10 is a device that estimates a state of each of at least one estimation target. Although described in detail later, each of the at least one estimation target is an imaging target whose state is to be estimated among at least one imaging target imaged using thermal imaging camera 20. For example, each of the at least one estimation target is a person or an animal. In the present case, the at least one estimation target is person 1 and state estimation device 10 estimates a state of person 1. For example, the state of each of the at least one estimation target is a state of sleep of each of the at least one estimation target. More specifically, the state of sleep indicates a depth of sleep, such as rapid eye movement (REM) sleep, non-REM sleep, arousal during sleep, or awake state. Other than these, the state of sleep may be, but not limited to, a pause in breathing during sleep that is caused by sleep apnea syndrome, for example. Other than the state of sleep, the state of each of the at least one estimation target may be, but not limited to, a state of healthcare of a person indoors, such as a state of activity (an amount of body movement) or a body temperature.

State estimation device 10 estimates the state of each of the at least one estimation target using thermal imaging camera 20. Thermal imaging camera 20 is a camera for capturing a thermal image. For example, the thermal image shows heat distribution. Thermal imaging camera 20 is disposed in a location where a thermal image of the at least one imaging target can be captured. In the present case, thermal imaging camera 20 is disposed above sleep place 2 where person 1 sleeps and captures a thermal image of person 1 sleeping in sleep place 2. For example, sleep place 2 is a bed. State estimation device 10 is communicatively connected to thermal imaging camera 20 and estimates, based on the thermal image captured using thermal imaging camera 20, the state of each of the at least one estimation target included among the at least one imaging target.

For example, state estimation device 10 is implemented by a processor and a memory.

State estimation device 10 has been described thus far.

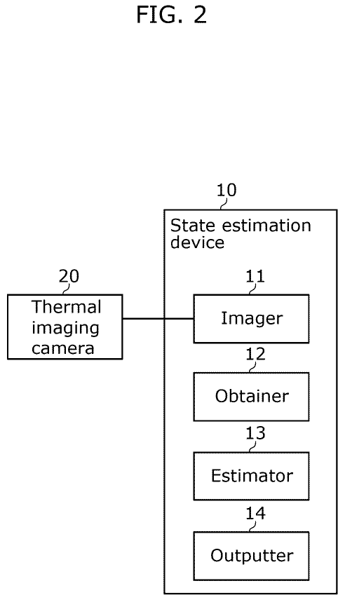

FIG. 2 is a block diagram illustrating a functional configuration of state estimation device 10 illustrated in FIG. 1. The functional configuration of state estimation device 10 is described with reference to FIG. 2.

As illustrated in FIG. 2, state estimation device 10 includes imager 11, obtainer 12, estimator 13, and outputter 14.

Imager 11 captures a thermal image of the at least one imaging target, each of which is a person or an animal, using thermal imaging camera 20. More specifically, each of the at least one imaging target is a person or an animal to be imaged using thermal imaging camera 20. For example, while each of the at least one imaging target is asleep, imager 11 captures a thermal image of the at least one imaging target during sleep using thermal imaging camera 20. For example, imager 11 captures a thermal image of the at least one imaging target using thermal imaging camera 20 at predetermined time intervals.

Obtainer 12 obtains identification information for identifying the at least one imaging target in the thermal image captured by imager 11. For example, the identification information includes information for estimating which is the at least one imaging target in the thermal image captured by imager 11. Moreover, the identification information includes information for estimating which is the at least one estimation target whose state is to be estimated among the at least one imaging target in the thermal image captured by imager 11.

For example, the identification information includes information that indicates a total number of the at least one imaging target. For example, if a thermal image of one imaging target is captured using thermal imaging camera 20, the identification information includes information indicating that the total number of the at least one imaging target is one. For example, if a thermal image of two imaging targets is captured using thermal imaging camera 20, the identification information includes information indicating that the total number of the at least one imaging target is two.

Furthermore, for example, the identification information includes: information indicating a total number of at least one person included among the at least one imaging target; and information indicating a total number of at least one animal included among the at least one imaging target. For example, if a thermal image of one person and one dog is captured using thermal imaging camera 20, the identification information includes: information indicating that the total number of the at least one person included among the at least one imaging target is one; and information indicating that the total number of the at least one animal included among the at least one imaging target is one. Furthermore, for example, if a thermal image of one person, one dog, and one cat is captured using thermal imaging camera 20, the identification information includes: information indicating that the total number of the at least one person included among the at least one imaging target is one; and information indicating that the total number of the at least one animal included among the at least one imaging target is two.

Furthermore, for example, the identification information includes information indicating a sleep location for each of the at least one estimation target. For example, the identification information includes information indicating an absolute sleep location for each of the at least one estimation target. For example, the identification information includes information indicating a relative sleep location for each of the at least one estimation target.

Furthermore, for example, the identification information includes information indicating a total number of the at least one estimation target. For example, if the at least one imaging target is one person and the state of this person is to be estimated, the identification information includes information indicating that the total number of the at least one estimation target is one. Furthermore, for example, if the at least one imaging target includes one person and one dog and the state of only the person is to be estimated among the person and the dog, the identification information includes information indicating that the total number of the at least one estimation target is one.

Furthermore, for example, the identification information includes: information indicating a total number of at least one person included among the at least one estimation target; and information indicating a total number of at least one animal included among the at least one estimation target. For example, if the state of one person and the state of one dog are to be estimated, the identification information includes: information indicating that the total number of the at least one person included among the at least one estimation target is one; and information indicating the total number of the at least one animal included among the at least one estimation target is one. Furthermore, for example, the state of one person, the state of one dog, and the state of one cat are to be estimated, the identification information includes: information indicating that the total number of the at least one person included among the at least one estimation target is one; and information indicating the total number of the at least one animal included among the at least one estimation target is two.

For example, the identification information is previously inputted by a user for instance and stored into state estimation device 10. Obtainer 12 obtains the identification information stored in state estimation device 10. Note that obtainer 12 may obtain the identification information stored in an external device of state estimation device 10, for example.

Estimator 13 estimates the state of each of the at least one estimation target among the at least one imaging target, based on the thermal image captured by imager 11 and the identification information obtained by obtainer 12. For example, estimator 13 identifies, based on the identification information, which is the at least one estimation target in the thermal image, and then estimates the state of each of the at least one estimation target based on a temperature for each of the at least one estimation target in the thermal image.

For example, if a total number of at least one section having a predetermined temperature or higher in the thermal image captured by imager 11 exceeds the total number of the at least one imaging target, estimator 13 preferentially identifies, as the at least one imaging target, a section having a higher temperature among the at least one section. For example, if the total number of the at least one section having the predetermined temperature or higher in the thermal image captured by imager 11 is three and the total number of the at least one imaging target is two, estimator 13 identifies a section having the highest temperature and a section having a second highest temperature as the at least one imaging target among the at least one section having the predetermined temperature or higher in the thermal image. For example, each of the at least one section having the predetermined temperature or higher in the thermal image is a collection of areas having the predetermined temperature or higher in the thermal image.

Furthermore, for example, estimator 13 preferentially identifies, as a person, an imaging target present in a predetermined location earlier among the at least one imaging target in the thermal image captured by imager 11. For example, if the at least one imaging target includes two persons and one animal, estimator 13 identifies, as persons, an imaging target that is present earliest in the predetermined location and an imaging target that is present next after the earliest imaging target, among the at least one imaging target in the thermal image captured by imager 11.

Furthermore, for example, estimator 13 identifies the at least one estimation target based on a location of the at least one imaging target in the thermal image captured by imager 11. For example, if the identification information includes the information indicating the absolute sleep location of the at least one estimation target, estimator 13 identifies which is the at least one estimation target in the thermal image based on the absolute sleep position of the at least one imaging target in the thermal image captured by imager 11.

Furthermore, for example, if the identification information includes the information indicating the relative sleep location of the at least one estimation target, estimator 13 identifies which is the at least one estimation target in the thermal image based on a positional relationship between the at least one imaging target in the thermal image captured by imager 11.

Outputter 14 outputs a result of the estimation by estimator 13. For example, outputter 14 outputs the result of the estimation to an analysis device that performs an analysis using the result of the estimation by estimator 13.

The functional configuration of state estimation device 10 has been described thus far.

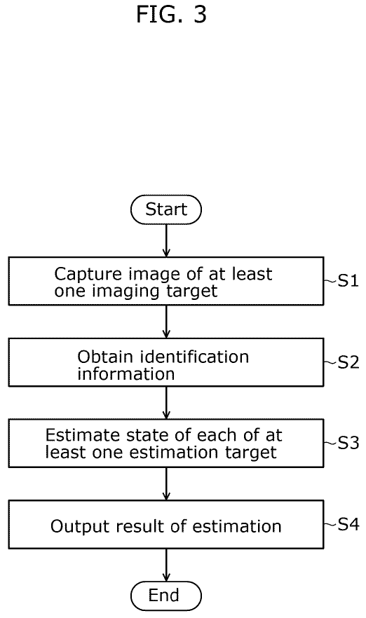

FIG. 3 is a flowchart illustrating an example of an operation performed by state estimation device 10 illustrated in FIG. 1. The example of the operation performed by state estimation device 10 is described with reference to FIG. 3.

As illustrated in FIG. 3, imager 11 captures a thermal image of the at least one imaging target, each of which is a person or an animal, using thermal imaging camera 20 (capturing) (Step S1).

After imager 11 captures the thermal image of the at least one imaging target, obtainer 12 obtains the identification information for identifying the at least one imaging target in the thermal image captured in the capturing (obtaining) (Step S2).

After obtainer 12 obtains the identification information, estimator 13 estimates the state of each of the at least one estimation target included among the at least one imaging target, based on the thermal image captured in the capturing and the identification information obtained in the obtaining (estimating) (Step S3).

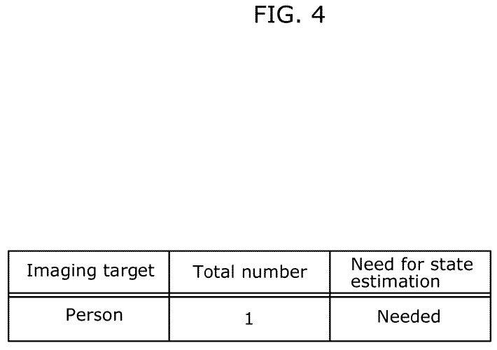

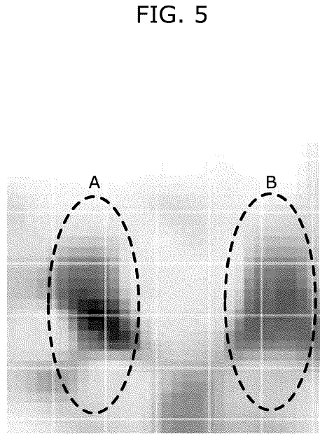

FIG. 4 is a table illustrating a first example of the identification information. FIG. 5 is a diagram illustrating a first example of the thermal image. A first example of state estimation performed by estimator 13 is described with reference to FIG. 4 and FIG. 5.

As illustrated in FIG. 4, the identification information in the present example includes information indicating: the imaging target is a person; the total number of imaging-target persons is one; and whether the state estimation on the person, who is the imaging target, is needed, or more specifically, whether the person, who is the imaging target, is an estimation target.

As described above, the identification information includes: the information indicating the total number of the at least one imaging target; the information indicating the total number of the at least one person included among the at least one imaging target; the information indicating the total number of the at least one estimation target; and the information indicating the total number of the at least one person included among the at least one estimation target. In the present example, the identification information indicates: the total number of the at least one imaging target is one; the total number of the at least one person included among the at least one imaging target is one; the total number of the at least one estimation target is one; and the total number of the at least one person included among the at least one estimation target is one.

As illustrated in FIG. 5, the thermal image captured in the capturing in the present example includes two sections having the predetermined temperature or higher (see sections A and B enclosed by dashed lines in FIG. 5). More specifically, the total number of the at least one section having the predetermined temperature or higher in the thermal image captured in the capturing in the present example is two.

For example, assume that the imaging target wakes after falling asleep and then falls back asleep in a location different from the location where the imaging target was initially sleeping. In this case, both the initial location where the imaging target was sleeping and the current location where the imaging target is sleeping have the predetermined temperature or higher in the thermal image captured in the capturing. To be more specific, the total number of the at least one section having the predetermined temperature or higher in the thermal image captured in the capturing is more than the total number of the at least one imaging target.

For example, if the total number of the at least one section having the predetermined temperature or higher in the thermal image captured in the capturing is more than the total number of the at least one imaging target, estimator 13 preferentially identifies the section having a higher temperature among the at least one section, as the at least one imaging target in the estimating.

In the present example, the total number of the at least one section having the predetermined temperature or higher in the thermal image captured in the capturing is two and the total number of the at least one imaging target is one. More specifically, the total number of the at least one section having the predetermined temperature or higher in the thermal image captured in the capturing is more than the total number of the at least one imaging target. Thus, estimator 13 identifies, as the at least one imaging target, the section having the higher temperature (see section A enclosed by the dashed line in FIG. 5) among the at least one section.

In the present example, the total number of the at least one imaging target is the same as the total number of the at least one estimation target. Thus, estimator 13 identifies the at least one imaging target as the at least one estimation target.

Estimator 13 estimates the state of the at least one estimation target based on a temperature of the at least one estimation target in the thermal image. For example, estimator 13 estimates the depth of sleep of the at least one estimation target.

The first example of the state estimation performed by estimator 13 has been described thus far.

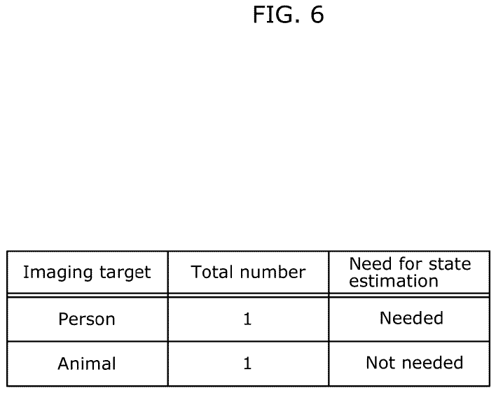

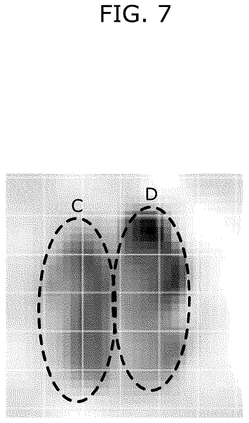

FIG. 6 is a table illustrating a second example of the identification information. FIG. 7 is a diagram illustrating a second example of the thermal image. A second example of the state estimation performed by estimator 13 is described with reference to FIG. 6 and FIG. 7.

As illustrated in FIG. 6, the identification information in the present example includes information indicating: the imaging targets are a person and an animal; the total number of imaging-target persons is one; the total number of imaging-target animal is one; whether the state estimation on the person, who is the imaging target, is needed, or more specifically, whether the person, who is the imaging target, is an estimation target; and whether the state estimation on the animal, which is the imaging target, is needed, or more specifically, whether the animal, which is the imaging target, is an estimation target.

As described above, the identification information includes: the information indicating the total number of the at least one imaging target; the information indicating the total number of the at least one person included among the at least one imaging target; the information indicating the total number of the at least one animal included among the at least one imaging target; the information indicating the total number of the at least one estimation target; and the information indicating the total number of the at least one person included among the at least one estimation target. In the present example, the identification information indicates: the total number of the at least one imaging target is two; the total number of the at least one person included among the at least one imaging target is one; the total number of the at least one animal included among the at least one imaging target is one; the total number of the at least one estimation target is one; and the total number of the at least one person included among the at least one estimation target is one.

As illustrated in FIG. 7, the thermal image captured in the capturing in the present example includes two sections having the predetermined temperature or higher (see sections C and D enclosed by dashed lines in FIG. 7). More specifically, the total number of the at least one section having the predetermined temperature or higher in the thermal image captured in the capturing in the present example is two.

For example, one of the at least one section corresponds to the person and the other one of the at least one one section corresponds to the animal.

For example, in the estimating, estimator 13 preferentially identifies, as the person, an imaging target that is positioned earlier in a predetermined location among the at least one imaging target in the thermal image captured in the imaging. For example, the predetermined location is a location, a thermal image of which is capturable by thermal imaging camera 20.

In the present example, in the thermal image captured in the capturing, the imaging target denoted as section D among the at least one imaging target is positioned earlier in the location, a thermal image of which is capturable by thermal imaging camera 20, than the imaging target denoted as section C among the at least one imaging target. Thus, estimator 13 identifies, as the person, the imaging target denoted as section D among the at least one imaging target and identifies, as the animal, the imaging target denoted as section C.

In the present example, the total number of the at least one estimation target is one and the total number of the at least one person included among the at least one estimation target is one. Thus, estimator 13 identifies the imaging target denoted as section D among the at least one imaging target, as the at least one estimation target.

Estimator 13 estimates the state of the at least one estimation target based on a temperature of the at least one estimation target in the thermal image. For example, estimator 13 estimates the depth of sleep of the at least one estimation target.

Note that, in the estimating, estimator 13 may preferentially identify, as the animal, the imaging target that is positioned earlier in the predetermined location among the at least one imaging target in the thermal image captured in the capturing, for example. Furthermore, estimator 13 may preferentially identify, as the animal, the imaging target having the highest temperature or a higher average temperature. This is because body temperatures of dogs and cats are higher than a body temperature of a person. In addition, a person wears clothes, and a surface temperature of the clothes, whose image is to be captured, is lower than a surface temperature of the skin. Furthermore, estimator 13 may estimate the state of the animal and the identification information may include information indicating the total number of the at least one animal included among the at least one estimation target.

The second example of the state estimation performed by estimator 13 has been described thus far.

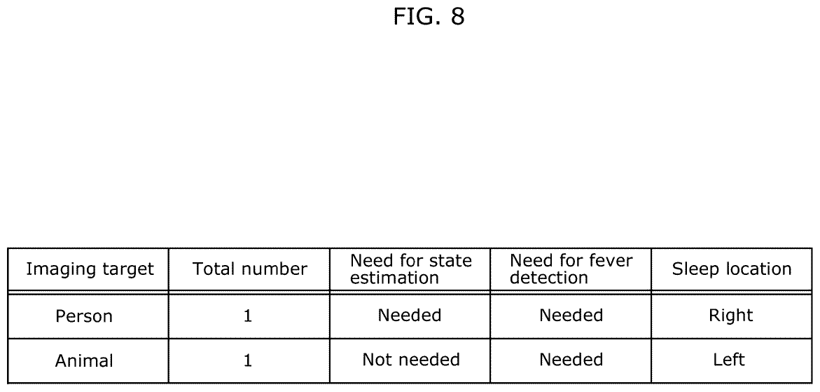

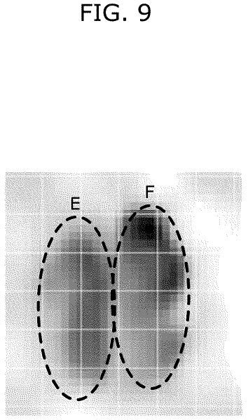

FIG. 8 is a table illustrating a third example of the identification information. FIG. 9 is a diagram illustrating a third example of the thermal image. A third example of the state estimation performed by estimator 13 is described with reference to FIG. 8 and FIG. 9.

As illustrated in FIG. 8, the identification information in the present example includes information indicating: the imaging targets are a person and an animal; the total number of imaging-target persons is one; the total number of imaging-target animal is one; whether the state estimation on the person, who is the imaging target, is needed, or more specifically, whether the person, who is the imaging target, is an estimation target; whether the state estimation on the animal, which is the imaging target, is needed, or more specifically, whether the animal, which is the imaging target, is an estimation target; whether fever detection on the person, who is the imaging target, is needed; whether fever detection on the animal, which is the imaging target, is needed; and sleep locations of the imaging targets.

As described above, the identification information includes: the information indicating the total number of the at least one imaging target; the information indicating the total number of the at least one person included among the at least one imaging target; the information indicating the total number of the at least one animal included among the at least one imaging target; the information indicating the total number of the at least one estimation target; the information indicating the total number of the at least one person included among the at least one estimation target; and the information indicating the sleep location for each of the at least one estimation target. In the present example, the identification information indicates: the total number of the at least one imaging target is two; the total number of the at least one person included among the at least one imaging target is one; the total number of the at least one animal included among the at least one imaging target is one; the total number of the at least one estimation target is one; the total number of the at least one person included among the at least one estimation target is one; and the person included among the at least one estimation target sleeps on the right side.

As illustrated in FIG. 9, the thermal image captured in the capturing in the present example includes two sections having the predetermined temperature or higher (see sections E and F enclosed by dashed lines in FIG. 9). More specifically, the total number of the at least one section having the predetermined temperature or higher in the thermal image captured in the capturing in the present example is two.

For example, one of the at least one section corresponds to the person and the other one of the at least one one section corresponds to the animal.

For example, in the estimating, estimator 13 identifies the at least one estimation target based on the location of the at least one imaging target in the thermal image captured in the capturing.

In the present example, in the thermal image captured in the capturing, the imaging target denoted as section F among the at least one imaging target is located on the right side of the imaging target denoted as section E among the at least one imaging target. Thus, estimator 13 identifies the imaging target denoted as section F among the at least one imaging target, as the at least one estimation target.

Estimator 13 estimates the state of the at least one estimation target based on a temperature of the at least one estimation target in the thermal image. For example, estimator 13 estimates the depth of sleep of the at least one estimation target.

For example, outputter 14 outputs an alert, based on a temperature of the at least one imaging target in the thermal image. For example, a normal temperature of the at least one imaging target is previously measured. Then, if the temperature of the at least one imaging target is higher than the normal temperature of the at least one imaging target, outputter 14 outputs an alert. To be more specific, assume that an average surface temperature of the imaging target is 33° C. and a root mean squared error (RMSE) is 0.5° C. in the last 10 days. Here, if the average surface temperature of the imaging target reaches 35° C., outputter 14 outputs the alert.

The third example of the state estimation performed by estimator 13 has been described thus far.

Referring back to FIG. 3, after estimator 13 estimates the state of each of the at least one estimation target, outputter 14 outputs a result of the estimation by estimator 13 (outputting) (Step S4).

The example of the operation performed by state estimation device 10 has been described thus far.

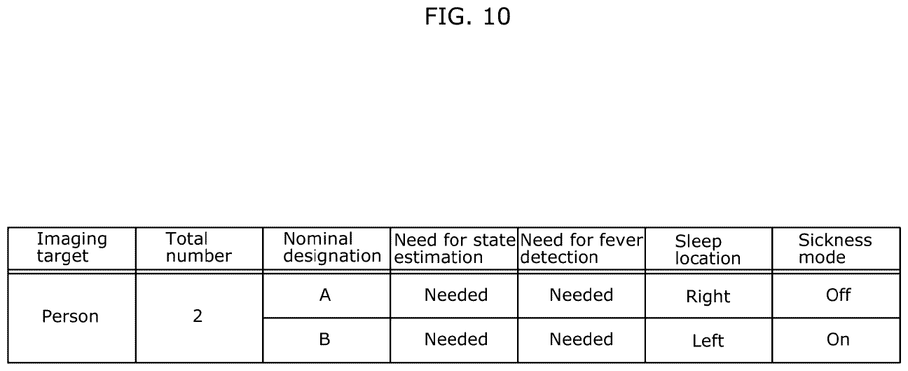

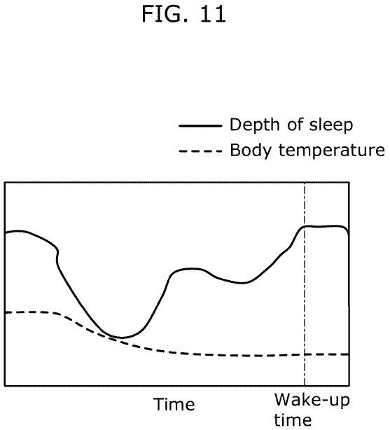

FIG. 10 is a table illustrating a fourth example of the identification information. FIG. 11 is a graph illustrating an example of another operation performed by state estimation device 10 illustrated in FIG. 1. The example of another operation performed by state estimation device 10 is described with reference to FIG. 10 and FIG. 11.

As illustrated in FIG. 10, the at least one imaging target includes persons referred to as A and B in the present example. B is sick and thus a sickness mode is set for B.

For example, estimator 13 estimates the depth of sleep of B based on the thermal image captured in the capturing and the identification information obtained in the obtaining. Moreover, outputter 14 notifies, in real time, A of the result of the estimation by estimator 13. This allows A to know that B is asleep and to be careful not to wake B, for example.

Furthermore, for example, outputter 14 notifies A of the temperature of B. This allows A to know whether the temperature of B is rising or falling.

Furthermore, for example, estimator 13 determines, based on the depth of sleep of B, whether B has woken up. When B is awake, outputter 14 notifies A that B has woken up. As illustrated in FIG. 11, if the depth of sleep of B is shallow while the body temperature of B is low, estimator 13 determines that B has woken up. A is to be notified when B is awake, and this allows A not to disturb the sleep of B. Moreover, this notification allows A, when B wakes up, to ask B about the condition of B and take care of a meal for B, for example.

State estimation device 10 and so forth according to Embodiment 1 have been described thus far.

A state estimation method according to Embodiment 1 includes: capturing a thermal image of at least one imaging target, each of which is a person or an animal, using thermal imaging camera 20 (Step S1); obtaining identification information for identifying the at least one imaging target in the thermal image captured in the capturing (Step S2); and estimating a state of each of at least one estimation target included among the at least one imaging target, based on the thermal image captured in the capturing and the identification information obtained in the obtaining (Step S3).

With this, the identification information facilitates the identification of the at least one imaging target in the thermal image. This facilitates the identification of the at least one estimation target included among the at least one imaging target in the thermal image. Thus, the state of the at least one estimation target can be estimated with ease and high accuracy.

Furthermore, by the state estimation method according to Embodiment 1, the estimating includes estimating a state of sleep of each of the at least one estimation target.

With this, the state of sleep of the at least one estimation target can be estimated with ease and high accuracy.

Furthermore, by the state estimation method according to Embodiment 1, the identification information includes information indicating a total number of the at least one imaging target.

With this, the total number of the at least one imaging target is used. This use further facilitates the identification of the at least one imaging target in the thermal image, and thereby further facilitates the identification of the at least one estimation target included among the at least one imaging target in the thermal image. Thus, the state of the at least one estimation target can be estimated with more ease and higher accuracy.

Furthermore, by the state estimation method according to Embodiment 1, if a total number of at least one section having a predetermined temperature or higher in the thermal image captured in the capturing exceeds the total number of the at least one imaging target, the estimating includes preferentially identifying, as the at least one imaging target, a section having a higher temperature among the at least one section.

With this, a section that does not correspond to the at least one imaging target in the thermal image is prevented from being estimated as the at least one imaging target. This further facilitates the identification of the at least one imaging target in the thermal image, and thereby further facilitates the identification of the at least one estimation target included among the at least one imaging target in the thermal image. Thus, the state of the at least one estimation target can be estimated with more ease and higher accuracy.

Furthermore, by the state estimation method according to Embodiment 1, the identification information includes: information indicating a total number of at least one person included among the at least one imaging target; and information indicating a total number of at least one animal included among the at least one imaging target.

With this, the total number of the at least one person and the total number of the at least one animal included among the at least one imaging target are used. This use further facilitates the identification of the at least one imaging target in the thermal image, and thereby further facilitates the identification of the at least one estimation target included among the at least one imaging target in the thermal image. Thus, the state of the at least one estimation target can be estimated with more ease and higher accuracy. Furthermore, by the state estimation method according to Embodiment 1, the estimating includes preferentially identifying, as the person, an imaging target that is positioned earlier in a predetermined location among the at least one imaging target in the thermal image captured in the capturing.

With this, the person captured in the thermal image can be easily estimated. This further facilitates the identification of the at least one imaging target in the thermal image, and thereby further facilitates the identification of the at least one estimation target included among the at least one imaging target in the thermal image. Thus, the state of the at least one estimation target can be estimated with more ease and higher accuracy.

Furthermore, by the state estimation method according to Embodiment 1, the identification information includes information indicating a sleep location for each of the at least one estimation target.

With this, the sleep location for each of the at least one estimation target is used. This use further facilitates the identification of the at least one imaging target in the thermal image, and thereby further facilitates the identification of the at least one estimation target included among the at least one imaging target in the thermal image. Thus, the state of the at least one estimation target can be estimated with more ease and higher accuracy.

Furthermore, by the state estimation method according to Embodiment 1, the estimating includes identifying the at least one estimation target based on a location of the at least one imaging target in the thermal image captured in the capturing.

With this, the location of the at least one imaging target in the thermal image further facilitates the identification of the at least one estimation target included among the at least one imaging target in the thermal image. Thus, the state of the at least one estimation target can be estimated with more ease and higher accuracy.

Furthermore, by the state estimation method according to Embodiment 1, the identification information includes information indicating a total number of the at least one estimation target.

With this, the total number of the at least one estimation target is used. This use further facilitates the identification of the at least one imaging target in the thermal image, and thereby further facilitates the identification of the at least one estimation target included among the at least one imaging target in the thermal image. Thus, the state of the at least one estimation target can be estimated with more ease and higher accuracy.

Furthermore, by the state estimation method according to Embodiment 1, the identification information includes: information indicating a total number of at least one person included among the at least one estimation target; and information indicating a total number of at least one animal included among the at least one estimation target.

With this, the total number of the at least one person and the total number of the at least one animal included among the at least one estimation target are used. This use further facilitates the identification of the at least one estimation target included among the at least one imaging target. Thus, the state of the at least one estimation target can be estimated with more ease and higher accuracy.

Furthermore, a state estimation device according to Embodiment 1 includes: imager 11 that captures a thermal image of at least one imaging target, each of which is a person or an animal, using thermal imaging camera 20; obtainer 12 that obtains identification information for identifying the at least one imaging target in the thermal image captured by imager 11; and estimator 13 that estimates a state of each of at least one estimation target included among the at least one imaging target, based on the thermal image captured by imager 11 and the identification information obtained by obtainer 12.

With this, the same operational advantage as the above-described state estimation method can be achieved.

### Embodiment 2

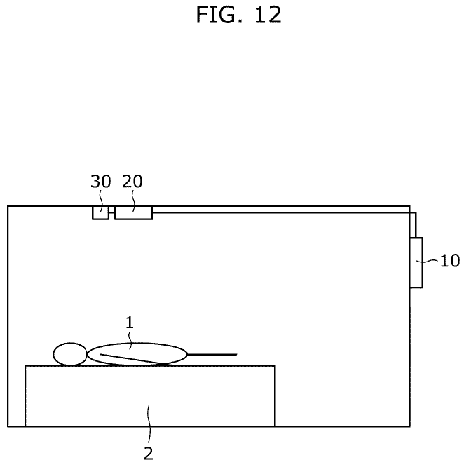

FIG. 12 is a diagram illustrating state estimation device 10 and so forth according to Embodiment 2. State estimation device 10 and so forth according to Embodiment 2 are described with reference to FIG. 12.

As illustrated in FIG. 12, state estimation device 10 according to Embodiment 2 is different from state estimation device 10 according to Embodiment 1 mainly in that a result of detection by fire detection sensor 30 is also obtained.

Estimator 13 estimates a state of at least one estimation target, based on the result of the detection by fire detection sensor 30. For example, if fire detection sensor 30 detects smoke, estimator 13 estimates that at least one estimation target is awake. This prevents estimator 13 from mistakenly estimating that the at least one estimation target is asleep although the at least one estimation target is smoking a cigarette instead of sleeping, for example.

State estimation device 10 and so forth according to Embodiment 2 have been described thus far.

### Embodiment 3

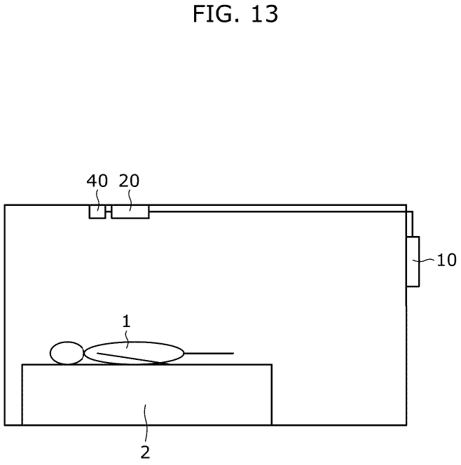

FIG. 13 is a diagram illustrating state estimation device 10 and so forth according to Embodiment 3. State estimation device 10 and so forth according to Embodiment 3 are described with reference to FIG. 13.

As illustrated in FIG. 13, state estimation device 10 according to Embodiment 3 is different from state estimation device 10 according to Embodiment 1 mainly in that a result of detection by illuminance sensor 40 is also obtained.

Estimator 13 estimates a state of at least one estimation target, based on the result of the detection by illuminance sensor 40.

For example, if illuminance detected by illuminance sensor 40 is a predetermined illuminance or lower, estimator 13 estimates that at least one estimation target is asleep. This allows estimator 13 to easily estimate that the at least one estimation target is asleep, for example. Thus, estimator 13 can easily estimate the state of sleep of the at least one estimation target.

Furthermore, for example, if the illuminance detected by illuminance sensor 40 is higher than the predetermined illuminance, estimator 13 estimates that the at least one imaging target is awake. This allows estimator 13 to easily estimate that the at least one estimation target is awake, for example. Thus, estimator 13 can easily estimate the awake state of the at least one estimation target.

Note, for example, that estimator 13 may obtain a signal indicating an operational status of a lighting device or a signal indicating an on-off state of a switch of the lighting device. Based on the signal, estimator 13 may estimate whether the at least one estimation target is asleep or awake.

State estimation device 10 and so forth according to Embodiment 3 have been described thus far.

### Embodiment 4

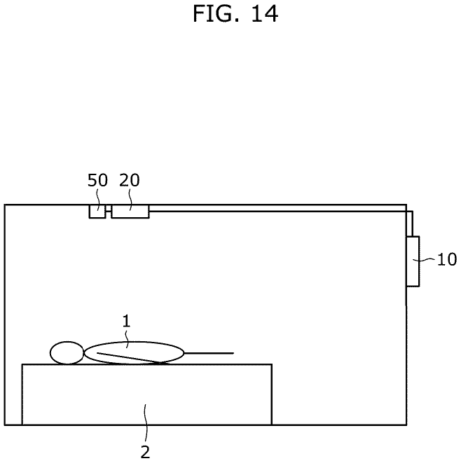

FIG. 14 is a diagram illustrating state estimation device 10 and so forth according to Embodiment 4. State estimation device 10 and so forth according to Embodiment 4 are described with reference to FIG. 14.

As illustrated in FIG. 14, state estimation device 10 according to Embodiment 4 is different from state estimation device 10 according to Embodiment 1 mainly in that lighting device 50 is controlled.

State estimation device 10 estimates a body movement of at least one estimation target from a thermal image, and controls lighting device 50 based on the body movement of the at least one estimation target.

For example, if estimating that the at least one estimation target is ready to sleep, state estimation device 10 dims down a light by controlling lighting device 50. This promotes sleep of the at least one estimation target.

Furthermore, for example, state estimation device 10 controls lighting device 50 based on time of day.

For example, if it is time for the at least one estimation target to wake up, state estimation device 10 dims up the light by controlling lighting device 50. This encourages the at least one estimation target to wake up.

Furthermore, for example, if the at least one estimation target goes to sleep in the daytime, state estimation device 10 dims up the light 10 to 30 minutes after dimming down the light. This prevents circadian rhythm disruption and enables more comfortable sleep.

Furthermore, for example, if estimating that the at least one estimation target has fallen off the bed, state estimation device 10 dims up the light by controlling lighting device 50. For example, if the at least one estimation target has stayed next to the bed for a predetermined period of time without turning on the light, state estimation device 10 determines that the at least one estimation target has fallen off the bed.

State estimation device 10 and so forth according to Embodiment 4 have been described thus far.

### Embodiment 5

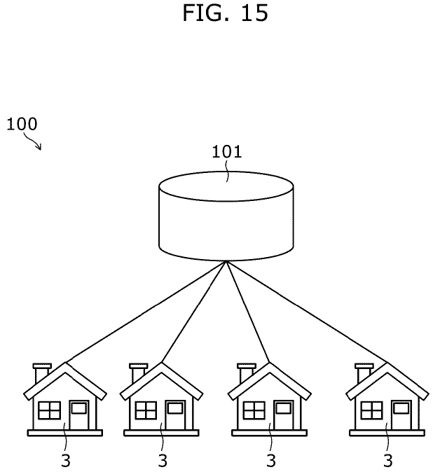

FIG. 15 is a diagram illustrating state management system 100 and so forth according to Embodiment 5. State management system 100 and so forth according to Embodiment 5 are described with reference to FIG. 15.

As illustrated in FIG. 15, state management system 100 according to Embodiment 5 includes: server 101; and a plurality of state estimation devices 10 (see FIG. 1 for example) provided for a plurality of residences 3, one state estimation device 10 being provided per one residence 3.

Each of the plurality of state estimation devices 10 transmits a result of estimation by estimator 13 to server 101.

Server 101 generates statistical information from the result of the estimation transmitted from each of the plurality of state estimation devices 10, and manages the statistical information. For example, server 101 generates statistical information on sleep by region. Furthermore, server 101 generates statistical information on sleep by age, for example. Furthermore, server 101 generates statistical information on sleep by event, for example.

Server 101 transmits the generated statistical information to each of the plurality of state estimation devices 10.

With this, a person living in a corresponding one of the plurality of residences 3 is able to know a bedtime status of a different person who lives nearby. This reminds the person not to cause trouble, such as noise, to the different person and go to sleep in accordance with the bedtime status of the different person. As a result, sleep efficiency increases for each region.

Furthermore, with this, when a percentage of sleepers in a specific region exceeds a predetermined threshold value, the gate to this region may be closed, for example. This leads to the prevention of crimes. As a result, sleep efficiency increases in this region.

Furthermore, with this, when a percentage of sleepers in a specific region exceeds a predetermined threshold value, lights of convenience stores and street lights in this region may be turned off, for example. This promotes energy conservation.

Furthermore, with this, when a percentage of sleepers in a specific region exceeds a predetermined threshold value, a road to this region may be closed, for example. This leads to the prevention of crimes. As a result, sleep efficiency increases in this region.

Furthermore, with this, when a percentage of sleepers in a specific region exceeds a predetermined threshold value, the maintenance of public systems of this region may be performed, for example. In this case, the maintenance can be performed without interfering with the lives of residents of this region, for example.

State management system 100 and so forth according to Embodiment 5 have been described thus far.

### Other Embodiments

Although a state estimation device according to one or more aspects of the present disclosure has been described based on embodiments, the present disclosure is not limited to these embodiments. Those skilled in the art will readily appreciate that embodiments arrived at by making various modifications to the above embodiments without materially departing from the scope of the present disclosure may be included within one or more aspects of the present disclosure.

For example, a result of estimation of bedtime and a result of estimation of wake-up time for each set imaging target may be informed to a user (the imaging-target person himself/herself, for example) on or after the following morning. In this case, the use of the bedtime and wake-up time facilitates distinction between a person and a pet (an animal). If the distinction between the person and the pet is incorrect, a correction may be made to improve distinction to be made on and after the following day.

Furthermore, for example, hot-cold feelings of a person or a pet may be estimated from a thermal image, and then air conditioning of the room may be controlled. To be more specific, based on the result of identification of the person and the pet, the air conditioning may be controlled based on the hot-cold feeling of the person instead of the pet. This promotes comfortable sleep of the person. For example, the hot-cold feeling can be estimated from a difference between a temperature of an area corresponding to the person (or animal) in the thermal image and a temperature around this area in the thermal image. However, the method for estimating the hot-cold feeling is not limited to this, and any method may be used. Note that the air conditioning may be controlled based on the hot-cold feeling of the pet, for example. Furthermore, if a plurality of persons are sleeping, the air conditioning may be set to be controlled based on the hot-cold feeling of a specific one of the plurality of persons, for example. Alternatively, the air conditioning may be controlled based on a feeling intermediate between the feelings of two persons, for example.

Furthermore, for example, the at least one imaging target may include a person and an animal, or may include two persons. If the at least one imaging target includes two persons, the at least one imaging target may be represented as person A and person B. Furthermore, for example, if the two persons are close to each other and thus difficult to distinguish between these persons, this section may be omitted from the state detection.

Each of the elements in each of the above embodiments may be configured in the form of an exclusive hardware product, or may be realized by executing a software program suitable for the element. Each of the elements may be realized by means of a program executing unit, such as a Central Processing Unit (CPU) or a processor, reading and executing the software program recorded on a recording medium such as a hard disk or semiconductor memory. Here, the software for realizing the above-described devices according to the embodiments is a program that causes a computer to execute each step included in the flowchart of FIG. 3.

The followings are also included in the present disclosure.

(1) At least one of the above-described devices may be a computer system including a microprocessor, a Read Only Memory (ROM), a Random Access Memory (RAM), a hard disk unit, a display unit, a keyboard, a mouse, and the like. The RAM or the hard disk unit holds a computer program. The microprocessor operates according to the computer program, thereby causing the constituent elements to execute their functions. Here, the computer program includes combinations of instruction codes for issuing instructions to the computer to execute predetermined functions.

(2) It should also be noted that a part or all of the constituent elements in at least one of the above-described devices may be implemented into a single Large Scale Integration (LSI). The system LSI is a super multi-function LSI that is a single chip into which a plurality of constituent elements are integrated. More specifically, the system LSI is a computer system including a microprocessor, a ROM, a RAM, and the like. The RAM holds a computer program. The microprocessor operates according to the computer program, thereby causing the system LSI to execute their functions.

(3) It should also be noted that a part or all of the constituent elements included in at last one of the above-described devices may be implemented into an Integrated Circuit (IC) card or a single module which is attachable to and removable from the device. The IC card or the module is a computer system including a microprocessor, a ROM, a RAM, and the like. The IC card or the module may include the above-described super multi-function LSI. The microprocessor operates according to the computer program to cause the IC card or the module to execute its functions. The IC card or the module may have tamper resistance.

(4) The present disclosure may be the above-described methods. The present disclosure may be a computer program realized by executing the methods using a computer, or may be digital signals including the computer program.

The present disclosure may be a computer-readable recording medium on which the computer program or the digital signals are recorded. Examples of the computer-readable recording medium are a flexible disk, a hard disk, a Compact Disc-Read Only Memory (CD-ROM), a magnetooptic disk (MO), a Digital Versatile Disc (DVD), a DVD-ROM, a DVD-RAM, a BD (Blu-ray(registered trademark) Disc), and a semiconductor memory. The present disclosure may be the digital signals recorded on the recording medium.

The present disclosure may be implemented by transmitting the computer program or the digital signals via an electric communication line, a wired or wireless communication line, a network represented by the Internet, data broadcasting, and the like.

It is also possible that the program or the digital signals may be recorded onto the recording medium to be transferred, or may be transmitted via a network or the like, so that the program or the digital signals can be executed by a different independent computer system.

### INDUSTRIAL APPLICABILITY

The state estimation device and so forth according to the present disclosure are applicable to, for example, a device that estimates a state of at least one estimation target.

### REFERENCE SIGNS LIST

10 state estimation device
11 imager
12 obtainer
13 estimator
14 outputter
100 state management system
101 server

1. A state estimation method comprising:
capturing a thermal image of at least one imaging target, each of which is a person or an animal, using a thermal imaging camera;
obtaining identification information for identifying the at least one imaging target in the thermal image captured in the capturing; and
estimating a state of each of at least one estimation target included among the at least one imaging target, based on the thermal image captured in the capturing and the identification information obtained in the obtaining.

2. The state estimation method according to claim 1,
wherein in the estimating, a state of sleep of each of the at least one estimation target is estimated.

3. The state estimation method according to claim 1,
wherein the identification information includes information indicating a total number of the at least one imaging target.

4. The state estimation method according to claim 3,
wherein when a total number of at least one section having a predetermined temperature or higher in the thermal image captured in the capturing exceeds the total number of the at least one imaging target, the estimating includes preferentially identifying, as the at least one imaging target, a section having a higher temperature among the at least one section.

5. The state estimation method according to claim 1,
wherein the identification information includes: information indicating a total number of at least one person included among the at least one imaging target; and information indicating a total number of at least one animal included among the at least one imaging target.

6. The state estimation method according to claim 5,
wherein the estimating includes preferentially identifying, as the person, an imaging target present in a predetermined location earlier among the at least one imaging target in the thermal image captured in the capturing.

7. The state estimation method according to claim 1,
wherein the identification information includes information indicating a sleep location of each of the at least one estimation target.

8. The state estimation method according to claim 7,
wherein the estimating includes identifying the at least one estimation target based on a location of each of the at least one imaging target in the thermal image captured in the capturing.

9. The state estimation method according to claim 1,
wherein the identification information includes information indicating a total number of the at least one estimation target.

10. The state estimation method according to claim 1,
wherein the identification information includes: information indicating a total number of at least one person included among the at least one estimation target; and information indicating a total number of at least one animal included among the at least one estimation target.

11. A state estimation device comprising:
an imager that captures a thermal image of at least one imaging target, each of which is a person or an animal, using a thermal imaging camera;
an obtainer that obtains identification information for identifying the at least one imaging target in the thermal image captured by the imager, and
an estimator that estimates a state of each of at least one estimation target included among the at least one imaging target, based on the thermal image captured by the imager and the identification information obtained by the obtainer.

12. A non-transitory computer-readable recording medium having recorded thereon a program for causing a computer to execute the state estimation method according to claim 1.
